(19) United States
(12) Patent Application Publication
(10) Pub. No.: US 2019/0150747 A1
(43) Pub. Date: May 23, 2019

(54) SPECTRAL EMISSION DEVICE FOR HEALTH EXAMINATION

(71) Applicants: Getwell Exercise & Health Technology (Shenzhen) Co., Ltd., Shenzhen City (CN); GETWELL HEALTH TECHNOLOGY (WUHU) CO. LTD., Wuhu City (CN)

(72) Inventors: Bin HUANG, Winnipeg (CA); Gong ZHANG, Winnipeg (CA); Zhikun YU, Shenzhen City (CN)

(21) Appl. No.: 16/177,650

(22) Filed: Nov. 1, 2018

(30) Foreign Application Priority Data
Nov. 23, 2017 (CN) 201711179263.3

(51) Int. Cl. A61B 5/00; F21V 19/00

(52) U.S. Cl. CPC A61B 5/0075; F21Y 2115/10; A61B 5/4519; F21V 19/0025

(57) ABSTRACT

The present invention discloses a spectral emission device for health examination including a base, power interface(s) and a plurality of LED lamps having different wavelengths, wherein the power interface(s) is/are disposed on one side of the base, and each of the power interfaces is correspondingly electrically connected to one of the LED lamps. The plurality of LED lamps are evenly distributed on an end surface of the base. The spectral emission device for health examination provides a light source for measuring SmO2, and a plurality of LEDs are incorporated into one package.

### FIELD

The present invention relates to the field of examination, and particularly to a spectral emission device for health examination.

### BACKGROUND

With regard to human muscle, the relative tendency of muscle oxygen saturation is calculated based on the difference between the reflection coefficient of oxyhemoglobin and of deoxyhemoglobin, to near-infrared light.

Nowadays, as this type of detection method requires various spectral emission devices to be designed, urgent problems to be solved, including how to carry out the spectrum emission and how to design the emission light source, occur.

### SUMMARY

It is an object of the present invention to provide a spectral emission device for health examination that provides a light source for measuring SmO2, wherein a plurality of LEDs are incorporated into one package.

In order to achieve the above object, the present invention provides a spectral emission device for health examination, comprising: a base, power interface(s), and a plurality of LED lamps having different wavelengths; wherein the power interface(s) is/are disposed on one side of the base, and each of the power interfaces is correspondingly electrically connected to one of the LED lamps; and said plurality of LED lamps are evenly distributed on an end surface of the base.

In some embodiments, the power interface comprises a notched anode port and a plurality of cathode ports, and one of the LED lamp is connected between any one of the cathode ports and the notched anode port, and each of the LED lamps has a different wavelength.

In some embodiments, the LED lamps have a quantity of five, and the wavelengths of each of the LED lamps are different from one another.

In some embodiments, the five LED lamps include: an LED lamp having a wavelength of 660 nm, an LED lamp having a wavelength of 730 nm, an LED lamp having a wavelength of 850 nm, an LED lamp having a wavelength of 810 nm, and an LED lamp having a wavelength of 940 nm.

In some embodiments, the cathode ports connected to said LED lamp having a wavelength of 660 nm, said LED lamp having a wavelength of 730 nm and said LED lamp having a wavelength of 850 nm are disposed on one side of the base; the cathode ports connected to said LED lamp having a wavelength of 810 nm and said LED lamp having a wavelength of 940 nm are disposed on other side of the base.

In some embodiments, the said notched anode port is disposed on same side of the base where said LED lamp having a wavelength of 810 nm and the said LED lamp having a wavelength of 940 nm are connected to the cathode ports.

According to the above technical solutions, the present invention achieves perfect control in one package, and provides a light source for product function and for measuring SmO2, and incorporates LEDs having different wavelengths into one package.

Other features and advantages of the invention will be described in detail in the following detailed description.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The drawings are intended to provide a further comprehension of the present invention, forming a part of the description, and in combination with the following detailed description to explain the present invention, but do not limit the scope of the present invention.

FIG. 1 is a schematic structural view showing a spectral emission device for health examination according to the present invention.

### REFERENCE LIST

1 base

2 power interface

3 LED lamp

4 notched anode port

5 cathode port of the LED lamp having a wavelength of 660 nm

6 cathode port of the LED lamp having a wavelength of 730 nm

7 cathode port of the LED lamp having a wavelength of 850 nm

8 cathode port of the LED lamp having a wavelength of 810 nm

9 cathode port of the LED lamp having a wavelength of 940 nm.

### DETAILED DESCRIPTION

The specific implementation of the present invention will be described in detail below with reference to the drawings. It is understood that the specific implementation described herein are merely illustrative and explanatory of the invention and are not intended to limit the present invention.

The present invention provides a spectral emission device for health examination, comprising: a base 1, power interface(s) 2 and a plurality of LED lamps 3 having different wavelengths; wherein said power interface(s) 2 is/are disposed on one side of base 1, and each power interface 2 is correspondingly electrically connected to one of the LED lamps 3; said plurality of the LED lamps 3 are evenly distributed on an end surface of the base 1.

According to the above technical solutions, the present invention achieves perfect control in one package, and provides a light source for product function and for measuring SmO2, and incorporates LEDs having different wavelengths into one package.

In a specific implementation of the present invention, the power interface 2 includes a notched anode port 4 and a plurality of cathode ports, and one of the LED lamps 3 is connected between any one of the cathode ports and the notched anode port 4, and each of the LED lamps 3 has a different wavelength.

In the above implementation, the LED lamps 3 can be powered on, and finally, the illumination of the lamps having different wavelengths can be realized.

In this implementation, the LED lamps 3 have a quantity of five, and the wavelengths of each of the LED lamps 3 are different. The product requires 5 wavelengths; the 5 wavelengths require a common anode drive; and achieve a perfect control in one package; provide a source of light for product function.

In this implementation, in order to realize detection of different wavelengths, on one aspect, the accuracy of detection is increased, and on the other aspect, the quantity of detections is increased, the five LED lamps 3 include: an LED lamp 3 having a wavelength of 660 nm, an LED lamp 3 having a wavelength of 730 nm, an LED lamp 3 having a wavelength of 850 nm, an LED lamp 3 having a wavelength of 810 nm, and an LED lamp 3 having a wavelength of 940 nm.

In this implementation, in order to achieve better detection effect, the cathode ports connected to said LED lamp 3 having a wavelength of 660 nm, said LED lamp 3 having a wavelength of 730 nm, and said LED lamp 3 having a wavelength of 850 nm are disposed on one side of the base 1; while the cathode ports connected to said LED lamp 3 having a wavelength of 810 nm and said LED lamp 3 having a wavelength of 940 nm are disposed on the other side of the base.

In this implementation, the notched anode port 4 is disposed on same side of the base 1 where the cathode ports is connected to the LED lamp 3 having a wavelength of 810 nm and the LED lamp 3 having a wavelength of 940 nm.

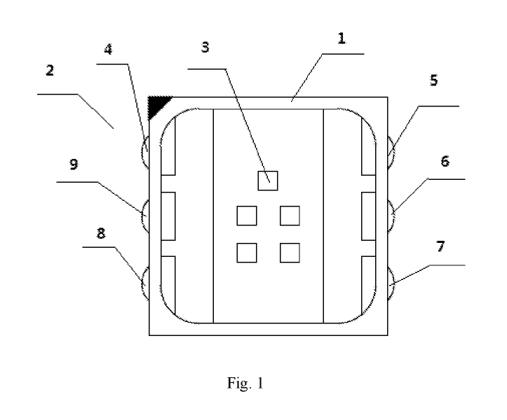

As shown in FIG. 1, the cathode port 5 of the LED lamp 3 having a wavelength of 660 nm, the cathode port 6 of the LED lamp 3 having a wavelength of 730 nm, the cathode port 7 of the LED lamp 3 having a wavelength of 850 nm, the cathode port 8 of the LED lamp 3 having a wavelength of 810 nm, and the cathode port 9 of the LED lamp 3 having a wavelength of 940 nm are respectively disposed on the base 1 as shown in FIG. 1.

Many modifications and variations of the present invention are possible in light of the above teachings. It is, therefore, to be understood that the invention may be practiced otherwise than as specifically described, and that the scope of the invention is defined by the claims.

It should be further noted that the specific technical features described in the above specific embodiments can be combined in any suitable manner if without contradiction. To avoid unnecessary repetition, the combination manner will not be otherwise described separately.

It is contemplated that all features of all claims and of all embodiments can be combined with each other, so long as such combinations would not contradict one another.

Listing of claims:

1. A spectral emission device for health examination, comprising:
a base;
power interface(s); and
a plurality of LED lamps (3) of different wavelengths,
wherein said power interface(s) is/are disposed on one side of the base, and each of the power interfaces is correspondingly electrically connected to one of the LED lamps, and said plurality of the LED lamps are evenly distributed on an end surface of the base.

2. The spectral emission device according to claim 1, wherein said power interface comprises: a notched anode port and a plurality of cathode ports, wherein one of the LED lamps is connected between any one of the cathode ports and the notched anode port, and each of said LED lamps has a different wavelength.

3. The spectral emission device according to claim 2, wherein said LED lamps have a quantity of five and the wavelengths of each of the LED lamps are different.

4. The spectral emission device according to claim 3, wherein the five LED lamps comprises: an LED lamp having a wavelength of 660 nm, an LED lamp having a wavelength of 730 nm, an LED lamp having a wavelength of 850 nm, an LED lamp having a wavelength of 810 nm and an LED lamp having a wavelength of 940 nm.

5. The spectral emission device according to claim 4, wherein the cathode ports connected to said LED lamp having a wavelength of 660 nm, said LED lamp having a wavelength of 730 nm, and said LED lamp having a wavelength of 850 nm are disposed on one side of the base; the cathode ports connected to said LED lamp having a wavelength of 810 nm and said LED lamp having a wavelength of 940 nm are disposed on other side of the base.

6. The spectral emission device according to claim 5, wherein the notched anode port is disposed on same side of said base where said LED light having a wavelength of 810 nm and said LED lamp having a wavelength of 940 nm are connected to the cathode ports.
